(19) United States
(12) Patent Application Publication
(10) Pub. No.: US 2018/0169365 A1
(43) Pub. Date: Jun. 21, 2018

(54) Intubating Neonatal Laryngeal Mask Airway

(71) Applicant: University of Washington, Seattle, WA (US)

(72) Inventors: Taylor Sawyer, Seattle, WA (US); Jonathan Posner, Seattle, WA (US); Stefan Foulstone, Seattle, WA (US); Sarah Owen, Seattle, WA (US); Neusha Farahani, Seattle, WA (US); Mark Trupiano, Seattle, WA (US)

(21) Appl. No.: 15/849,408

(22) Filed: Dec. 20, 2017

Related U.S. Application Data
(60) Provisional application No. 62/437,154, filed on Dec. 21, 2016.

(51) Int. Cl. A61M 16/04; A61M 16/06

(52) U.S. Cl. CPC A61M 16/0488; A61M 16/0409; A61M 16/06; A61M 16/0431; A61M 16/0415; A61M 16/0411; A61M 16/0434

(57) ABSTRACT

An intubating neonatal laryngeal mask airway includes an elongated body having a palate side and a lingual side. A cuff is coupled to an elongated body opposite a distal end of the elongated body. The cuff includes a concavity positioned opposite the palate side of the elongated body and between a guide sleeve and an esophageal plug. At least one ventilation lumen traverses the elongated body from the distal end to the cuff. The at least one ventilation lumen is in fluid communication with at least one ventilation port. A guide channel is longitudinally positioned on the lingual side of the elongated body. The guide channel is operatively aligned to a guide sleeve. An imaging device port is positioned within the concavity.

### CROSS-REFERENCE TO RELATED APPLICATIONS

This non-provisional patent application claims priority and benefit under 35 U.S.C. § 119(e) to U.S. Provisional application Ser. No. 62/437,154, filed on Dec. 21, 2016 and incorporated herein by reference in its entirety.

### BACKGROUND

Endotracheal intubation is a procedure in which a tube is passed through the mouth, past the vocal cords, and into the trachea. This is done for a variety of purposes including mechanical ventilation and delivery of anesthesia. In adults, intubation is a high volume procedure done before and after surgery. With neonates, intubation becomes more challenging due to the small size of the airway, the anterior positioning of the vocal cords, and the lack of color indication by the vocal cords. This problem is exacerbated with premature infants, who sometimes only weigh 500 grams. Another challenge with neonatal intubation compared to that of the adult intubation is that the time scale for intubation is much shorter. In general, neonatal intubation is only used in critical and lifesaving scenarios, where adult intubation is generally performed in a much more controlled environment.

Of the four million babies born in the United States every year, about 10% of them will require some form of oxygen delivery post birth, with one of the ways being intubation. One study found that one in every 500 babies born will require intubation. Although intubation is a widely used in adults, the first time success rates are extremely low in neonates. An “attempt” is defined as the placement of the laryngoscope into the infant's mouth and includes a cut off time of 30 seconds. The 30-second time limit per attempt is considered the gold standard of care. Originally, the gold standard was a 20-second limit per attempt, but it was found unachievable even by experienced physicians. Many studies have been conducted to analyze how low the neonatal intubation success rates are. A study conducted at the University of California, San Diego Medical Center conducted nearly 2700 intubations over the course of the study with 169 different pediatric interns, residents, and fellows. This study found that the average success rate 36% for all operators. Success rate did increase with a higher training level. Pediatric interns had a success rate of about 25% while second and third year fellows had reached just over a 50% success rate. This 50% success rate is also seen for attending neonatologists.

The most common form of neonatal intubation is by direct laryngoscopy, in which a laryngoscope is used to move the tongue and epiglottis out of the way, and create a direct line of sight to the cords, as well as a clear pathway for the tube to enter the airway. Variations of the laryngoscopy method include video laryngoscopy in which small video camera is strategically placed on the laryngoscope to provide a better visualization of the cords. Another variation, Airtrag™, uses fiber optic pathways in an attempt to create better visuals of the airway. Another device used in neonatal resuscitation is a laryngeal mask airway (LMA) which creates a seal by inflating a cuff around the opening at the larynx. This device is useful for mechanical ventilation, because it naturally disposes in the proper location. There are variations of LMAs that allow for intubation, but the traditional LMA does not require a tube placement, which makes it a temporary ventilation solution, as leaving the LMA in the airway complicates the access to the stomach and restricts blood flow to the brain. Intubating LMAs have been developed, but most do not facilitate the removal of the LMA after intubation. C-Trach™, an intubating LMA device with a camera and monitor, does allow for LMA removal after intubation, however, the process involves several steps, and is not easy to implement.

Another device that is less commonly used is a video stylet. A stylet is used to give endotracheal (“ET”) tubes more rigidity and shape, and in this case a camera at the tip of the stylet allows for visualization of the airway. However, using this device alone makes maneuvering around the anatomy more time-consuming and difficult as it does not have its structure designed for this function.

Due to this low success rate, the intubation procedure can require several attempts from a handful of different operators. Depending on the risk to the infant, the intern or resident will generally be allowed two or three attempts before the next operator is able to try. If it proves to be an especially difficult intubation, after about five people have attempted the intubation, the attending neonatologist is called into perform the intubation.

The low success rate of neonatal intubation can lead to a higher number of attempts on the infant resulting in irritation and inflammation. Other complications from intubation include bradycardia (slow heart rate), hypoxia (oxygen desaturation), apnea (interruption in breathing), and even death. A need therefore exists for a medical instrument which allows for oxygen delivery in 30 seconds or less, facilitates intubation, and that is compatible with different, pre-existing intubation tube sizes, in order to increase success rate among practitioners, especially for those with limited training and practice.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an isometric view of a neonatal laryngeal mask airway 100 in accordance with one embodiment.

FIG. 2 illustrates a front view of a neonatal laryngeal mask airway 100 in accordance with one embodiment.

FIG. 3 illustrates a lateral view of a neonatal laryngeal mask airway 100 in accordance with one embodiment.

FIG. 4 illustrates a neonatal laryngeal mask airway 100 in accordance with one embodiment.

FIG. 5 illustrates a cross sectional view of a neonatal laryngeal mask airway 100 in accordance with one embodiment.

FIG. 6 illustrates a cross sectional view of a cuff 600 in accordance with one embodiment.

FIG. 7 illustrates an isometric view of a neonatal laryngeal mask airway 816 with an attached ventilation connector in accordance with one embodiment.

FIG. 8 illustrates a lateral cross sectional view of a neonatal pharynx 800 of with a neonatal laryngeal mask airway 816 installed in accordance with one embodiment.

FIG. 9 illustrates an isometric view of a neonatal laryngeal mask airway 100 with an endotracheal tube in accordance with one embodiment.

FIG. 10 illustrates an isometric view of a neonatal laryngeal mask airway 1000 in accordance with one embodiment.

FIG. 11 illustrates a neonatal laryngeal mask airway 1000 in accordance with one embodiment.

FIG. 12 illustrates a method 1200 of intubating a patient with the neonatal laryngeal mask air way in accordance with one embodiment.

### DETAILED DESCRIPTION

In one embodiment, a neonatal laryngeal mask airway includes an elongated body having a palate side and a lingual side. A cuff is coupled to an elongated body opposite a distal end of the elongated body. The cuff includes a concavity positioned opposite the palate side of the elongated body and between a guide sleeve and an esophageal plug. At least one ventilation lumen traverses the elongated body from the distal end to the cuff. The at least one ventilation lumen is in fluid communication with at least one ventilation port. A guide channel is longitudinally positioned on the lingual side of the elongated body. The guide channel is operatively aligned to a guide sleeve. An imaging device port is positioned within the concavity.

The at least one ventilation lumen and the at least one ventilation port may include a pair of ventilation lumens and a pair of ventilation ports, respectively. The neonatal laryngeal mask airway may further include the imaging device port being positioned between the pair of ventilation ports, and the imaging device port being proximally positioned to the guide sleeve.

The imaging device port may be operatively coupled to an imaging cable traversing the elongated body through an imaging cable lumen. The lingual side of the elongated body further includes raised lateral ridges longitudinally traverse the lingual side of the elongated body forming the guide channel. In some configurations, the imagine device within the imaging device port may communicate imaging information to a display device through a wireless communication system (e.g., Bluetooth, WiFi, etc.,).

The material construction of the relative stiffness of the neonatal laryngeal mask airway is greater than relative stiffness of an intubation tube. The neonatal laryngeal mask airway is provided with a material construction allowing for the neonatal laryngeal mask airway to maintain its shape during insertion into a patient's pharynx and form a seal with the cuff without deformation of the at least one ventilations lumens. The neonatal laryngeal mask airway also has the flexibility to curve down the patient's throat in order to reduce potential damage to the soft tissue in the area. Comparatively, the rigidity (i.e., stiffness) of the neonatal laryngeal mask airway is slightly greater allowing the neonatal laryngeal mask airway to maintain its shape while the intubation tube traversing the guide channel and guide sleeve when it is being positioned within the patient's trachea.

In one embodiment, a method of intubating a patient with the neonatal laryngeal mask airway involves inserting a neonatal laryngeal mask airway into the pharynx of a patient, with a lingual side of the neonatal laryngeal mask airway positioned towards the patient's lingual surface. A cuff of the neonatal laryngeal mask airway is introduced into the larynx, forming a seal. At least one ventilation port on the cuff is aligned with the patient's trachea. Oxygenated air is supplied to the patient through the at least one ventilation port through a ventilation device by way of the at least one ventilation lumen. An endotracheal tube is inserted through the guide channel towards a guide sleeve of the cuff. The endotracheal tube traverses through the guide sleeve and into the trachea. Delivery of the oxygenated air is switched from the at least one ventilation port to the endotracheal tube, and the neonatal laryngeal mask airway is removed from the patient's mouth.

The method of intubating a patient with the neonatal laryngeal mask airway device may further involve utilizing an imaging device positioned within an imaging device port in the cuff to align the endotracheal tube with the patient's trachea and observe it pass between the vocal cords and into the trachea.

A neonatal laryngeal mask airway may include an elongated body having an outer surface, a proximal portion, a distal portion, and a longitudinal length, the distal portion of the body configured for extending into the throat of a patient during use, the body defining at least one oxygen delivery lumen extending along the longitudinal length and configured for enabling passage of sufficient oxygen along the longitudinal length of the body from a source external to the patient to ventilate the patient during use. A guide track is defined along the longitudinal length of the outer surface of the body, the guide track configured for receiving an endotracheal tube. A cuff is disposed along the distal portion of the body, the cuff configured for placement at a target location around the opening of the patient's larynx to provide a seal with the patient's larynx during use. An imaging subsystem disposed along the distal portion of the body may be utilized for visualizing a region in proximity to the distal portion of the body. The cuff is formed from a soft material. The imaging subsystem includes a camera and a light source both configured for coupling to a display disposed external to the patient. The light source may include at least one light emitting diode. The at least one oxygen delivery lumen may include two oxygen delivery lumens. The guide track may be formed to receive an endotracheal tube while the distal portion of the body is disposed in the patient's throat without interrupting the passage of oxygen along the length of the body.

The neonatal laryngeal mask airway is configured for being removed from the patient subsequent to receiving the endotracheal tube without necessitating a corresponding removal, or partial removal, of the endotracheal tube from the patient's trachea and without interrupting the passage of oxygen along the length of the body.

The at least one oxygen delivery lumen may have a rounded transverse cross section. The body may be formed from semi-rigid material and may include at least one bend that may range between 75° and 120° degrees allowing it to conform to a target location for the distal end that lies beneath the patient's epiglottis.

Ventilating a patient involves inserting the intubating neonatal laryngeal mask airway into the patient's throat, extending an endotracheal tube along the guide track of the neonatal laryngeal mask airway and into the patient's trachea, and removing the neonatal laryngeal mask airway while leaving the endotracheal tube in place within the patient's trachea.

An imaging subsystem and video display may be utilized to view the region in proximity to the distal portion of the body.

In one embodiment, the ventilation lumen(s) provide a flow of approximately 1.5 liters/min at a pressure not exceeding 20 mm of water. The lumens may take on any geometry as long as they maintain this flow without exceeding this pressure.

The intubating neonatal laryngeal mask airway device is a substantial improvement over the current standard of care, which is face mask ventilation and intubation via direct laryngoscopy. The intubating neonatal laryngeal mask airway device provides an effective means of ventilation as an LMA, and also allows for direct tracheal intubation through the LMA—when needed. The intubating neonatal laryngeal mask airway device is also a substantial improvement over the currently available neonatal LMAs that allow for tracheal intubation. Available LMAs that allow tracheal intubation require the operator to insert the endotracheal tube through the single airport of the LMA to perform intubation. This blocks air flow to the lungs, and interrupts ventilation of the lungs, while the endotracheal tube is being inserted. In neonates this interruption of ventilation can led to dangerous drops in the blood oxygen levels. Using current intubating LMAs, once the endotracheal tube is placed in the trachea the LMA is pulled out over the top of the endotracheal tube. This is risky in small infants because the maneuver could easily dislodge the endotracheal tube. In a neonate, the endotracheal tube is only inserted 8-10 cm to be positioned in the trachea. Thus, even small movement of the tube (1-2 cm) could dislodge it.

An intubating neonatal laryngeal mask airway device provides the benefits of a tradition laryngeal mask airway (LMA) device, and overcomes the limitations of currently available neonatal ‘intubating LMAs’. The neonatal intubating LMA includes several innovative design features to allow an easy transition from LMA ventilation to endotracheal tube ventilation, without the need to remove the LMA and perform direct laryngoscopy. The neonatal intubating LMA provides an endotracheal tube track that allows easy placement of the endotracheal tube into the trachea without removing the LMA. A video imaging system allowing visual confirmation that the endotracheal tube is in the trachea. A dual oxygen delivery ports that provide continuous ventilation during intubation, avoiding pauses in ventilation during intubation.

The track of intubating neonatal laryngeal mask airway device allows the intubating neonatal laryngeal mask airway device to be slid out from under—instead of over the top—of the endotracheal tube. This is a significant improvement over the current method. This method should decrease the chance of endotracheal tube dislodgement in small infants. The ability of the intubating neonatal laryngeal mask airway device to deliver continuous ventilation to the lungs is the most significant advantage. This feature is not seen in any adult or neonatal LMA device. The features of the intubating neonatal laryngeal mask airway device limit dangerous drops in blood oxygen levels that occur during neonatal intubation with current intubating LMAs and direct laryngoscopy. The intubating neonatal laryngeal mask airway device makes neonatal intubation safer than it is now, and will improve patient care.

The neonatal laryngeal mask airway device is compatible with neonatal sized ET tubes and caps, and can be used with standard ventilators. The inner diameter of intubating neonatal laryngeal mask airway device is larger than that of the neonatal ET tubes and thus provides similar amounts of airflow given the same amount of pressure. The camera utilized in intubating neonatal laryngeal mask airway device has a focal length of <2 mm and features a strong light source, providing clear visualization of airway. However, the camera placement may be optimized to provide the most convenient view of the cords. The intubating neonatal laryngeal mask airway device may include an inflatable cuff.

The utilized camera may have a focal length smaller than 7 mm and should fit the dimension constraints of the design. In addition to being small, high in short distance resolution, and short in focal length, these cameras are cost effective as they are designed and manufactured to be ultimately disposable. The utilized camera may be equipped with LED light sources and may measure 3×5×8 mm in size and come with a flexible cable attached to a USB port.

The elongated body and the cuff may all be molded and fabricated as a single piece. These components each may be formed from materials such as PDMS, silicone platinum, urethane, and polyurethane. Polyurethane may provide the fitting material for the cuff as it provides suitable rigidity and is resistant to tearing. Degassing may be utilized during the molding process to eliminate air bubbles, and a polyurethane release agent may be utilized to facilitate the removal of cured polyurethane from the mold. The shape of the camera port may be made rectangular to properly house a rectangular-shaped camera, if utilized.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

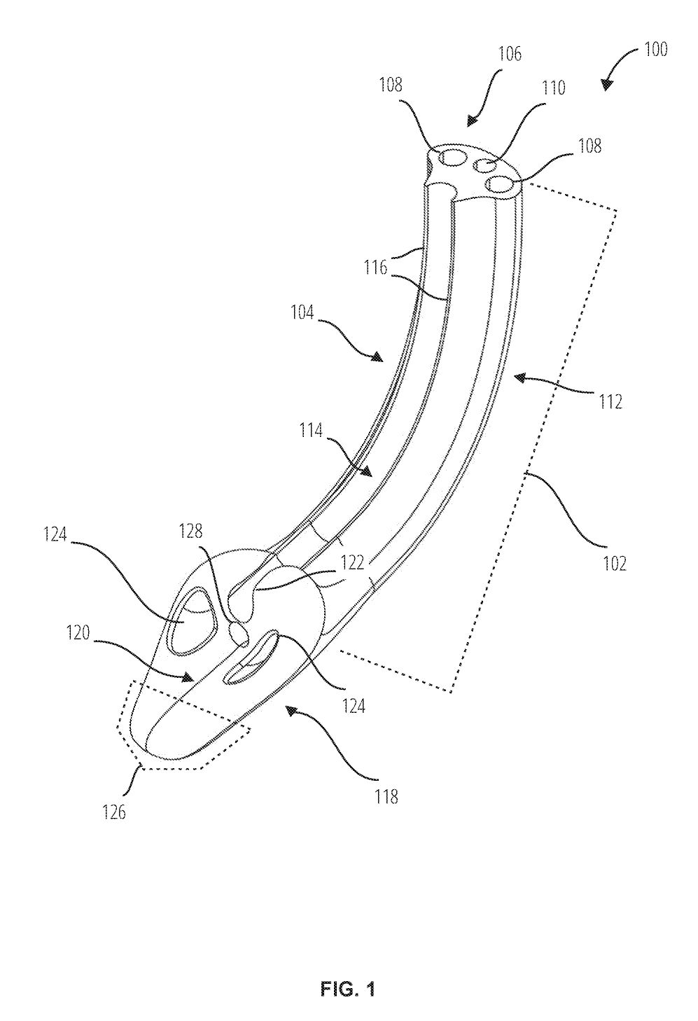

Various illustrative embodiments of the invention will now be described in reference to the drawings. Referencing FIG. 1, a neonatal laryngeal mask airway 100 is shown in an isometric view. The neonatal laryngeal mask airway 100 comprises an elongated body 102 coupled to a cuff 118. The elongated body 102 comprises a lingual side 104, a palate side 112, and a distal end 106 positioned opposite the cuff 118 along the length of the elongated body 102. The lingual side 104 and the palate side 112 are positioned opposite one another along the length of the elongated body 102. The elongated body 102 comprises a curvature adapted for extending the cuff 118 down a patient's throat following insertion. The lingual side 104 is proximal to the radial point of the curvature and is the portion of the elongated body 102 that contacts or is proximal to the lingual portion of a patient's mouth. The palate side 112 is the portion of the elongated body 102 that contacts or is proximal to the palate portion of the patient's mouth. The at least one ventilation lumen 108 and an imaging cable lumen 110 longitudinally traverses the elongated body 102 towards the cuff 118.

The cuff 118 comprises at least one ventilation port 124, an imaging device port 128, and a guide sleeve 122. The at least one ventilation lumen 108 is in fluid (e.g., gaseous) communication with the at least one ventilation port 124 such that oxygenated air passes through the at least one ventilation lumen 108 and exits through the at least one ventilation port 124. The imaging device port 128 may comprise an imaging device communicably coupled to a imaging device cable through the imaging cable lumen 110.

The cuff 118 in this example is slightly teardrop shaped in appearance and with a slant curving towards the palate side 112. The widest portion of the cuff 118 is proximal to lingual side 104 and tapers away approaching the palate side 112. An esophageal plug 126 is positioned on the distal portion of the cuff 118 opposite the placement of the guide sleeve 122. A concavity 120 is located in the cuff 118 between the guide sleeve 122 and the esophageal plug 126. The imaging device port 128 is disposed within the concavity 120, between the ventilation ports 124. The imaging device port 128 operatively disposes an imaging device towards a patient's trachea permitting visibility and visual confirmation of the positioning of the endotracheal tube within the patient's trachea.

A guide channel 114 is formed on the lingual side 104 by raised lateral ridges 116 extending away from the palate side 112. The guide channel 114 is adapted to direct an endotracheal tube down the patient's throat as it is inserted. The guide channel 114 is operatively aligned with the guide sleeve 122. During insertion of an endotracheal tube, the guide channel 114 directs the endotracheal tube to traverse through the guide sleeve 122. The guide sleeve 122 then directs the endotracheal tube towards the patient's trachea.

When the neonatal laryngeal mask airway 100 is introduced into a patient's pharynx, the shape of the cuff 118 seals the larynx, preventing airflow through the esophagus. The at least one ventilation port 124 is operatively disposed towards the trachea 804 permitting ventilation through mechanical or manual means. An endotracheal tube may be inserted along the guide channel 114 and through the guide sleeve 122 and into the trachea to secure a more permanent air supply. The position of the guide sleeve 122 directs the endotracheal tube past the epiglottis and through the trachea. Insertion of the endotracheal tube may occur while the patient is receiving oxygenated air through the at least one ventilation port 124. After the endotracheal tube is installed, the ventilation through the neonatal laryngeal mask airway 100 is switched to the endotracheal tube, and the neonatal laryngeal mask airway 100 is removed from the patient's pharynx.

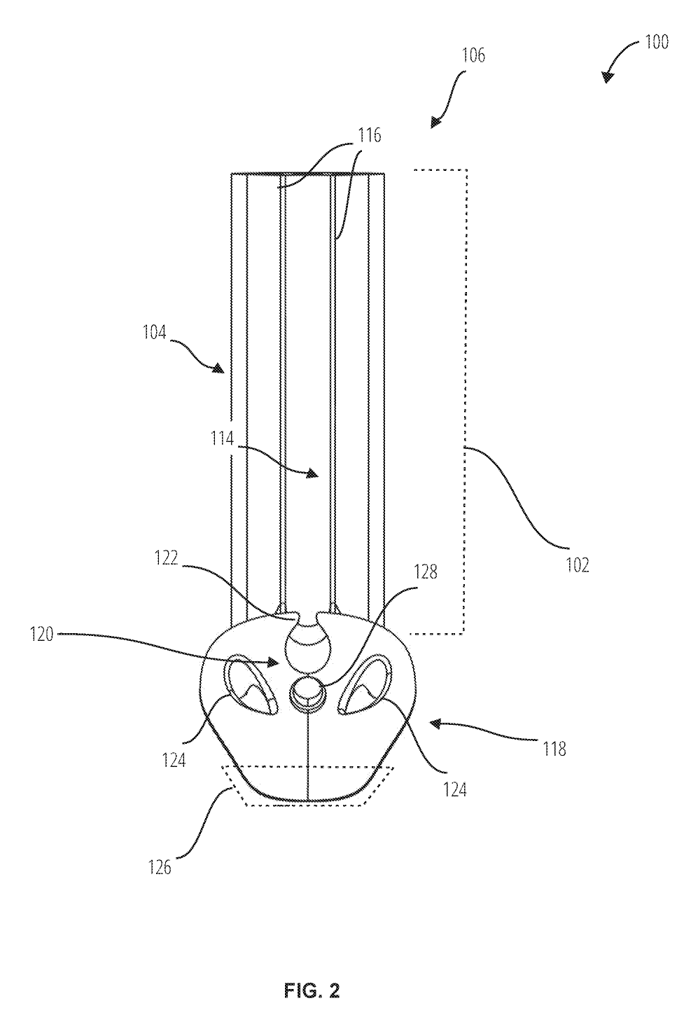

Referencing FIG. 2, the neonatal laryngeal mask airway 100 is shown in a front view. Shown in this view are the elongated body 102, lingual side 104, distal end 106, guide channel 114, raised lateral ridges 116, cuff 118, concavity 120, guide sleeve 122, at least one ventilation port 124, esophageal plug 126, and imaging device port 128.

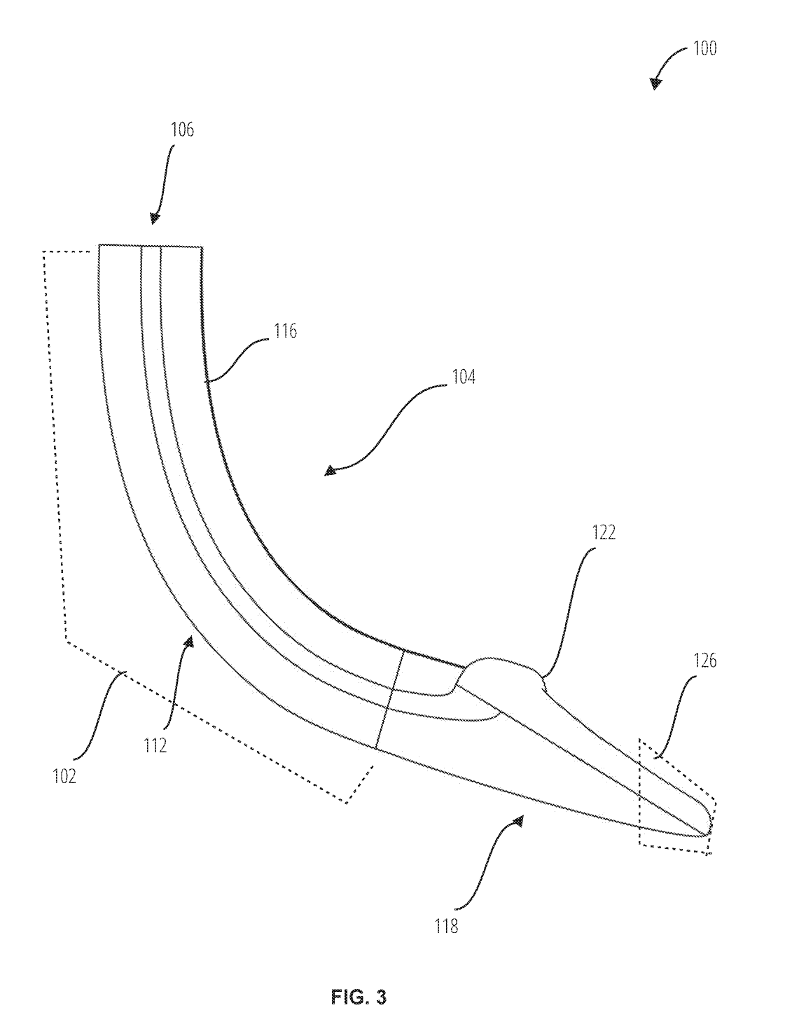

Referencing FIG. 3, the neonatal laryngeal mask airway 100 is shown in a lateral view. Shown in this view are the elongated body 102, lingual side 104, distal end 106, palate side 112, raised lateral ridges 116, cuff 118, guide sleeve 122, and an esophageal plug 126.

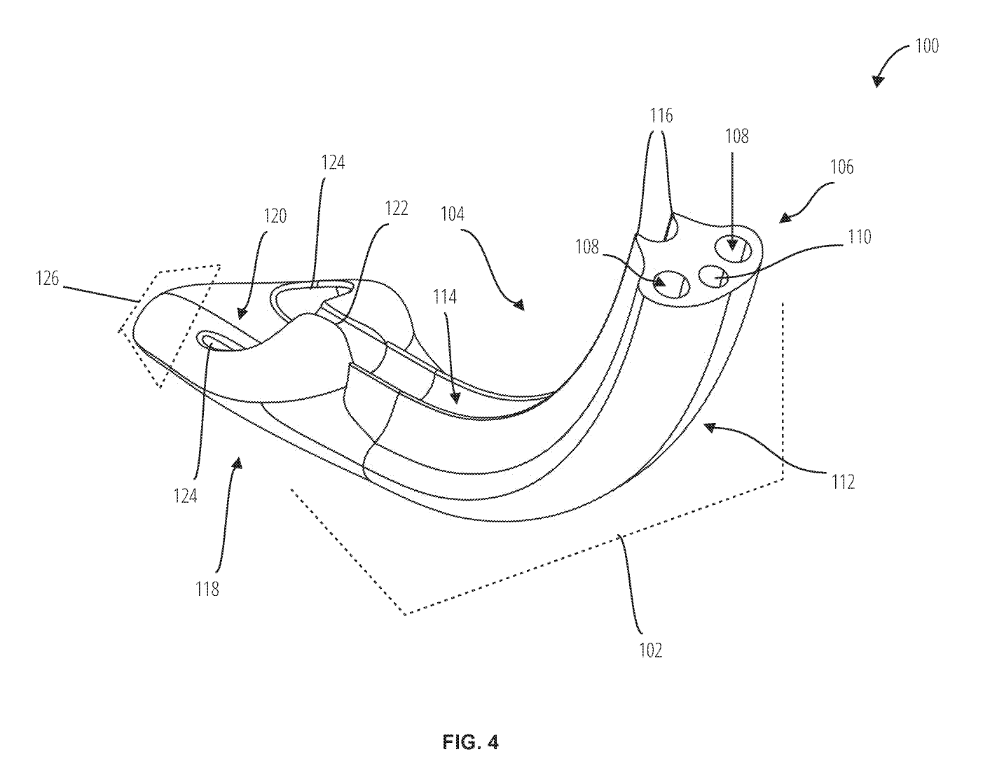

Referencing FIG. 4, the neonatal laryngeal mask airway 100 is shown in a rear isometric view. Shown in this view are the elongated body 102, lingual side 104, distal end 106, the ventilation lumen 108, the imaging cable lumen 110, the palate side 112, the guide channel 114, the raised lateral ridges 116, the cuff 118, the concavity 120, the guide sleeve 122, the ventilation port 124, and the esophageal plug 126.

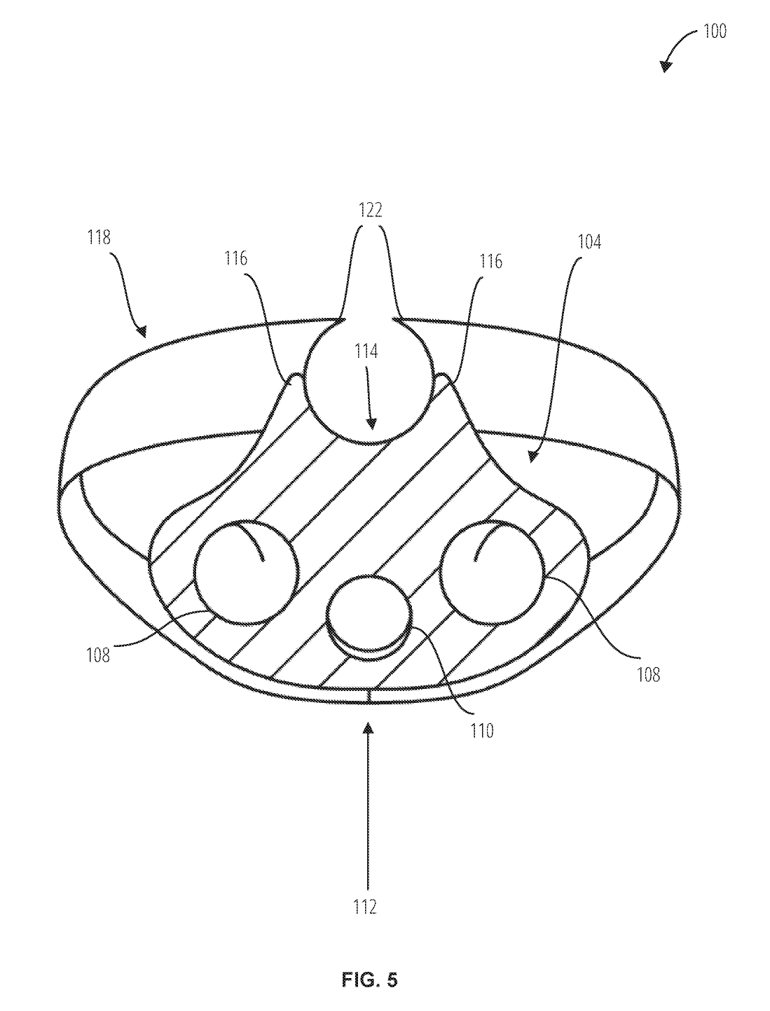

Referencing FIG. 5, the neonatal laryngeal mask airway 100 is shown in a cross sectional view. Shown in this view are the lingual side 104, the ventilation lumen 108, the imaging cable lumen 110, the palate side 112, the guide channel 114, the raised lateral ridges 116, the cuff 118, and the guide sleeve 122. The ventilation lumen 108 has a bilateral distribution through the length of the elongated body. The imaging cable lumen 110 is positioned between the at least one ventilation lumen 108 when in a bilateral distribution.

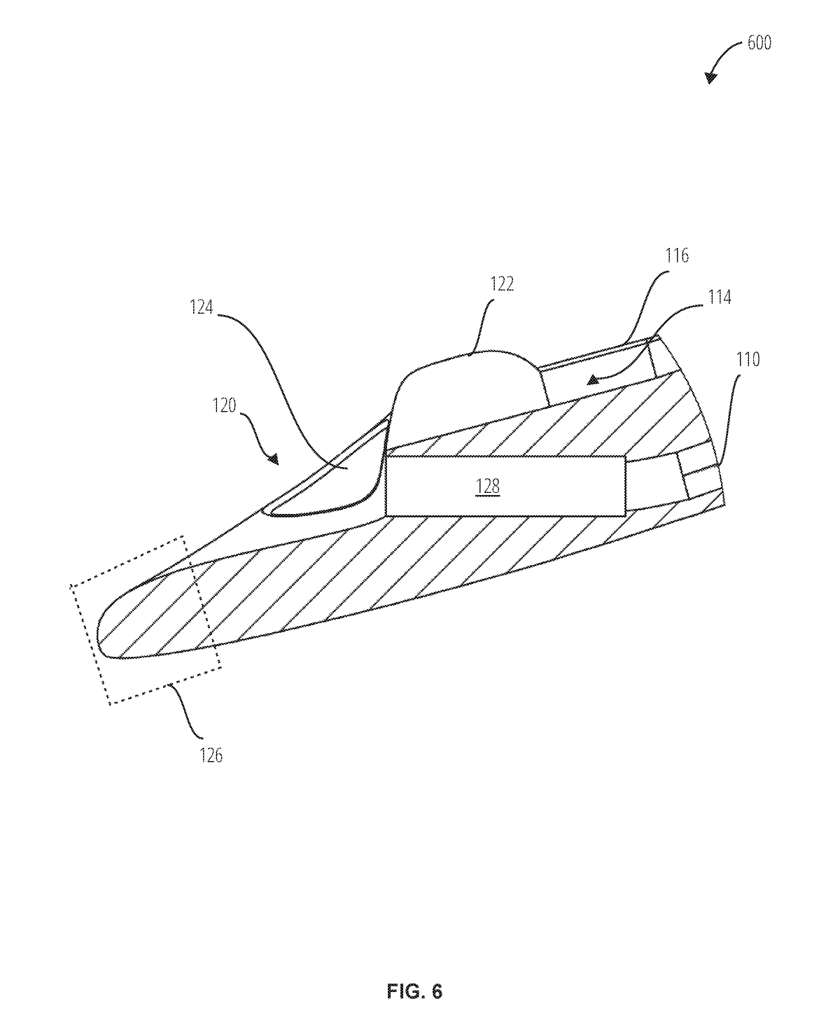

Referencing FIG. 6, a cuff 600 embodiment is shown in a cross sectional view. This view of the cuff 600 shows the imaging cable lumen 110, the guide channel 114, the raised lateral ridges 116, the concavity 120, the guide sleeve 122, the ventilation port 124, the esophageal plug 126, and the imaging device port 128.

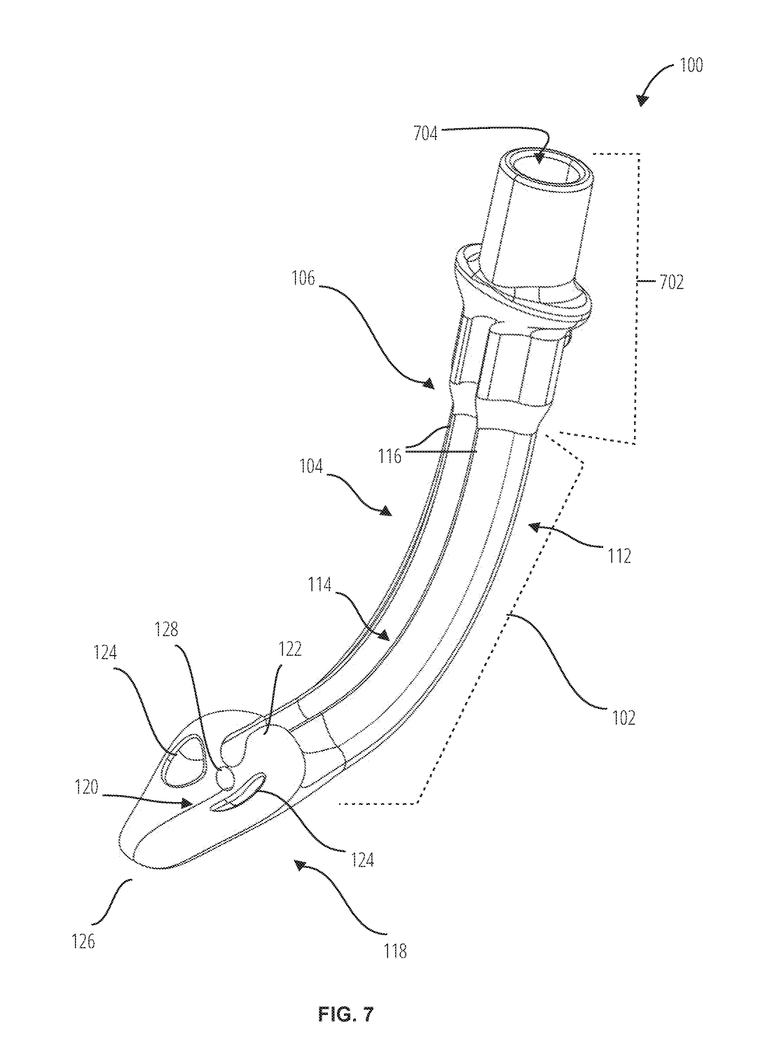

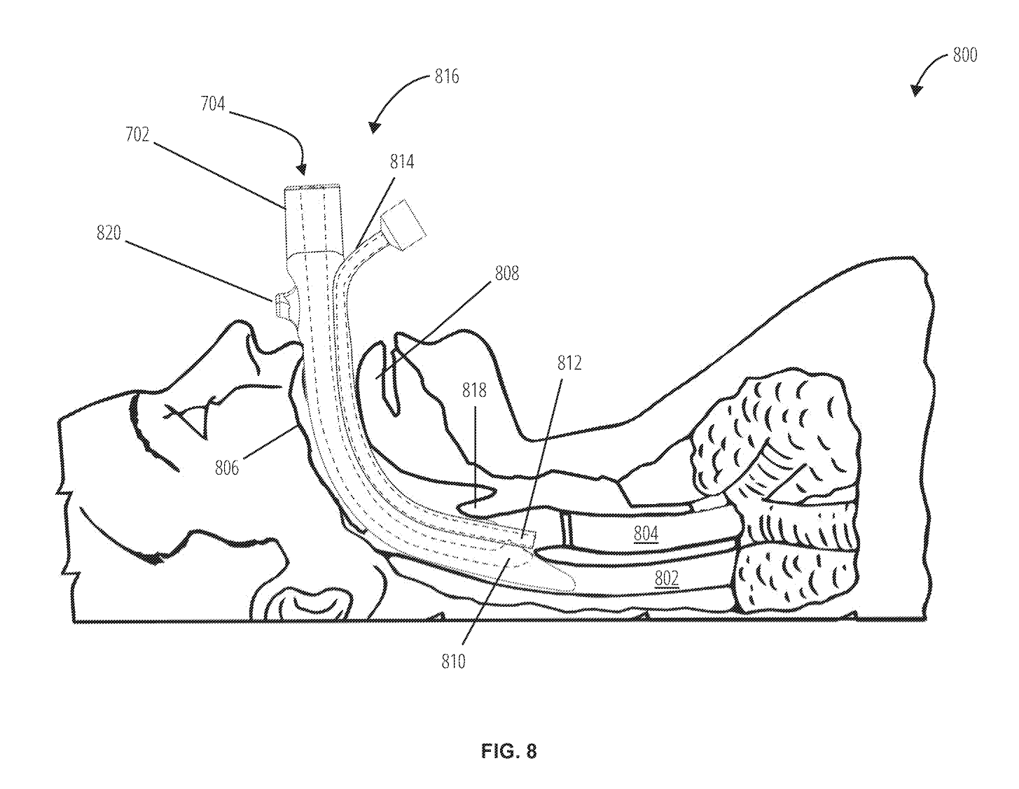

Referencing FIG. 7, the neonatal laryngeal mask airway 100 is shown in an isometric view with an attached ventilation connector. Shown in this view are the elongated body 102, lingual side 104, distal end 106, palate side 112, guide channel 114, raised lateral ridges 116, cuff 118, concavity 120, guide sleeve 122, ventilation port 124, esophageal plug 126, imaging device port 128, and a ventilation connector 702. This view shows the attachment of a ventilation connector 702 to the distal end 106 of the elongated body 102. The ventilation connector 702 provides connections for a manual or mechanical ventilation device through the large opening on the top. A ventilation connector port 704 of the ventilation connector 702 is in fluid communication with the at least one ventilation lumen while blocking airflow into the imaging cable lumen. An imaging cable port 820, shown in FIG. 8, positioned on the rear of the ventilation connector 702 provides a communicable coupling for the imaging device cable to traverse into the imaging cable lumen.

Referencing FIG. 8, a lateral cross sectional view of a neonatal pharynx 800 is shown with a 700 installed. The neonatal pharynx 800 comprises an esophagus 802, a trachea 804, a palate 806, epiglottis 818, and a tongue 808. A neonatal laryngeal mask airway 100 installed in a neonatal pharynx 800 comprises at least one ventilation lumen 810, an endotracheal tube lumen 812, an endotracheal tube 814, and a ventilation connector 702 comprising an imaging cable port 820 and a ventilation connector port 704.

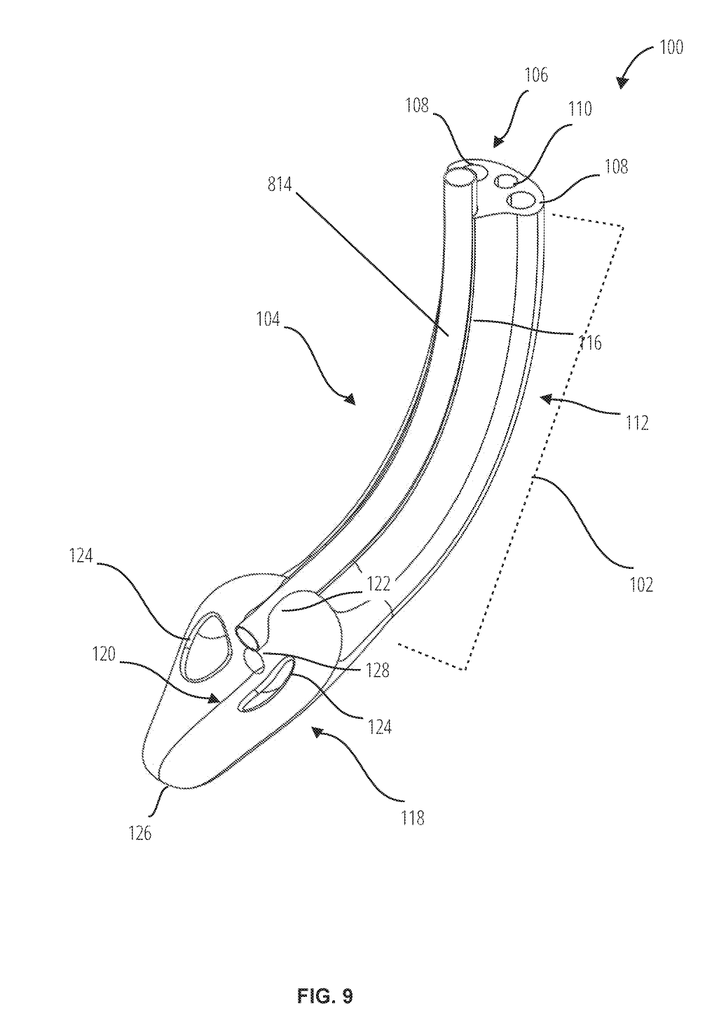

Referencing FIG. 9, the neonatal laryngeal mask airway 100 is shown in an isometric view with an endotracheal tube. Shown in this view are the elongated body 102, lingual side 104, distal end 106, ventilation lumen 108, imaging cable lumen 110, palate side 112, raised lateral ridges 116, cuff 118, concavity 120, guide sleeve 122, ventilation port 124, esophageal plug 126, imaging device port 128, and endotracheal tube 814. This view depicts the insertion of the endotracheal tube 814 through the guide channel traversing through the guide sleeve 122 as part of the intubation of the patient. The imaging device port 128 is disposed to view the insertion of the endotracheal tube 814 into the trachea, allowing for a medical professional to make corrections if it is out of alignment.

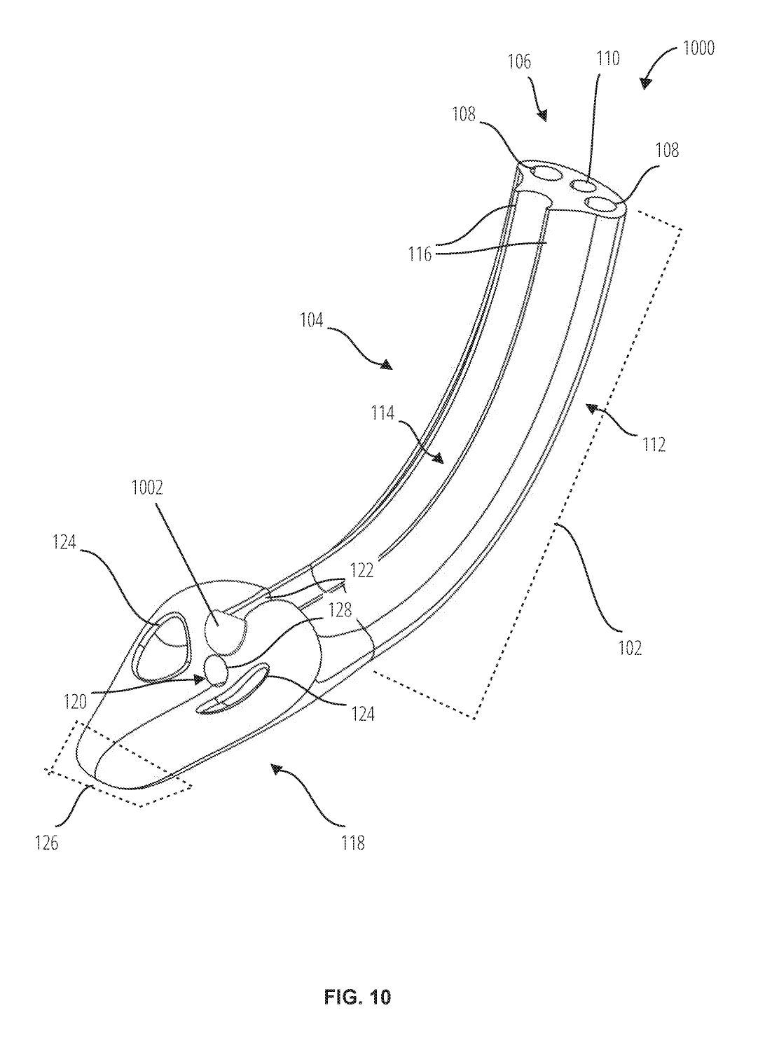

Referencing FIG. 10, the neonatal laryngeal mask airway 1000 is shown in a view depicting the elongated body 102, lingual side 104, distal end 106, ventilation lumen 108, imaging cable lumen 110, palate side 112, guide channel 114, raised lateral ridges 116, cuff 118, concavity 120, guide sleeve 122, ventilation port 124, esophageal plug 126, imaging device port 128, and a guide channel flap 1002. The guide channel flap 1002 is a moveable flap that occludes the guide channel 114 allowing the cuff 118 to form a complete seal around the larynx. This view shows the guide channel flap 1002 positioned in front of the guide sleeve 122 and the guide channel 114 blocking oxygenated air from the at least one ventilation port 124 from escaping through the opening of the guide sleeve 122. The guide channel flap 1002 may pivot out of the way of an endotracheal tube that is being guided through the channel.

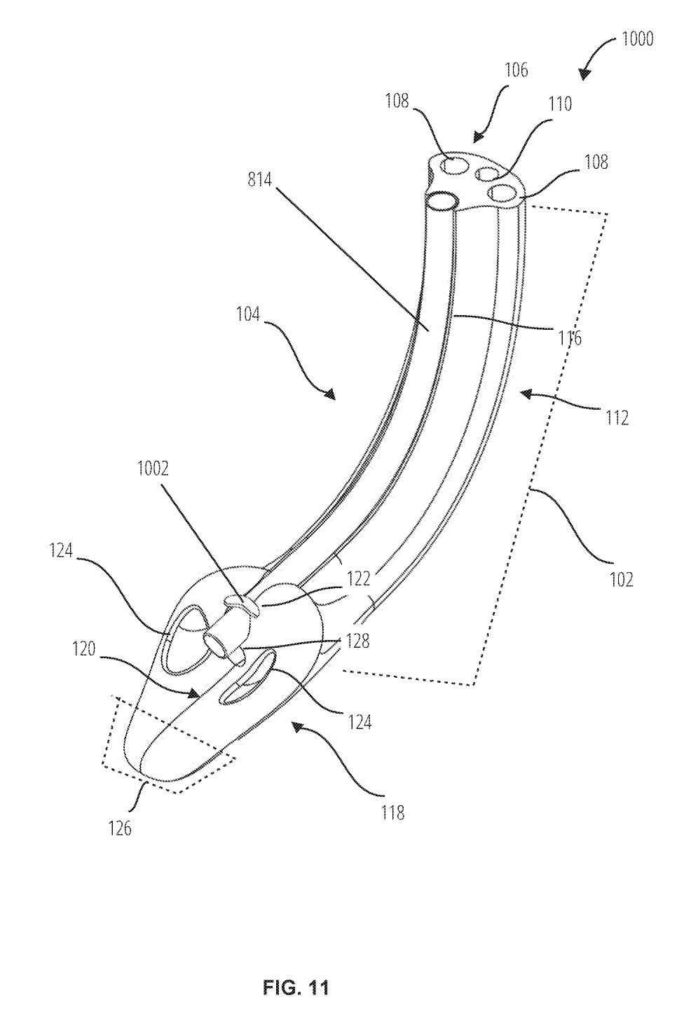

Referencing FIG. 11, the neonatal laryngeal mask airway 1000 is depicted in a view that shows the elongated body 102, lingual side 104, distal end 106, ventilation lumen 108, imaging cable lumen 110, palate side 112, raised lateral ridges 116, cuff 118, concavity 120, guide sleeve 122, ventilation port 124, esophageal plug 126, imaging device port 128, and endotracheal tube 814. This view shows the guide channel flap 1002 pivoted out of the way of the endotracheal tube 814 as it passes through the guide sleeve 122 after being guided through the guide channel.

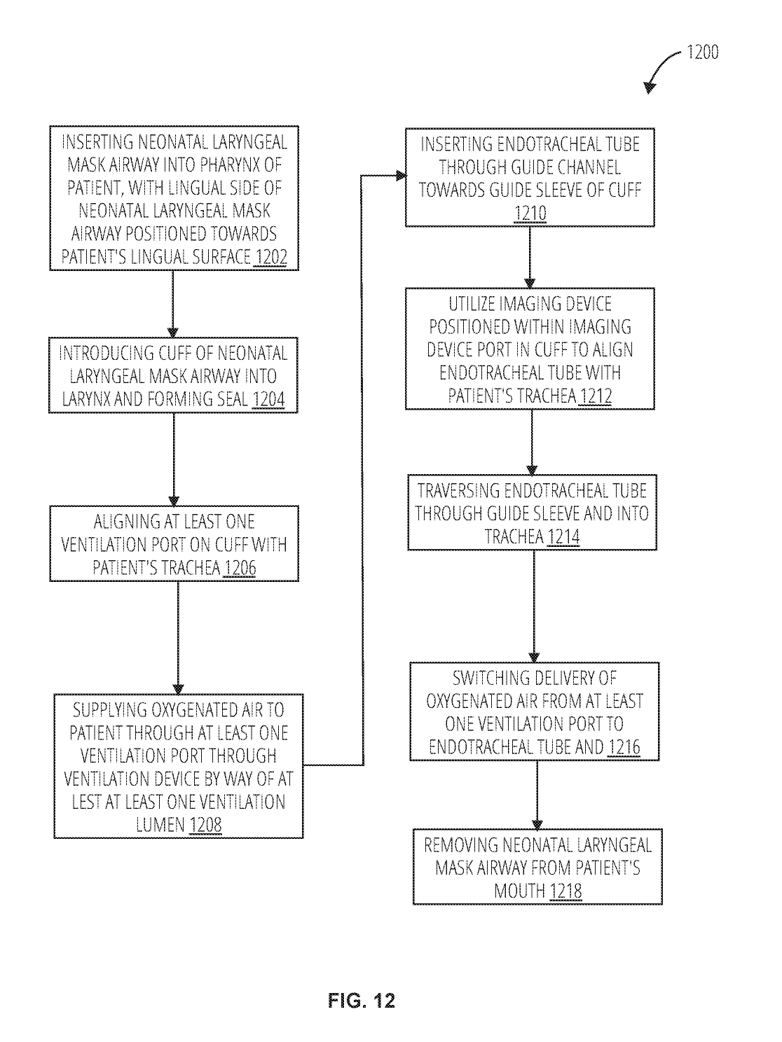

Referencing FIG. 12, a method 1200 of intubating a patient with the neonatal laryngeal mask air way 100 involves inserting a neonatal laryngeal mask airway 100 into the pharynx of a patient, with a lingual side of the neonatal laryngeal mask airway 100 positioned towards the patient's lingual surface (block 1202). In block 1204, the cuff of the neonatal laryngeal mask airway 100 is introduced into the larynx, forming a seal. In block 1206, at least one ventilation port on the cuff is aligned with the patient's trachea. In block 1208, oxygenated air is supplied to the patient through the at least one ventilation port through a ventilation device by way of the at least at least one ventilation lumen. In block 1210, an endotracheal tube is inserted along the guide channel towards a guide sleeve of the cuff. In block 1212, an imaging device positioned within an imaging device port in the cuff is utilized to align the endotracheal tube with the patient's trachea. In block 1214, the endotracheal tube is traversed through the guide sleeve and into the trachea. In block 1216, delivery of the oxygenated air is switched from the at least one ventilation port to the endotracheal tube. In block 1218, the neonatal laryngeal mask airway 100 is removed from the patient's mouth. As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. A neonatal laryngeal mask airway comprising:
an elongated body comprising a palate side and a lingual side;
a cuff coupled to the elongated body, opposite a distal end of the elongated body;
the cuff comprising a concavity positioned opposite the palate side of the elongated body and between a guide sleeve and an esophageal plug;
at least one ventilation lumen traversing the elongated body from the distal end to the cuff;
the at least one ventilation lumen being in fluid communication with at least one ventilation port;
a guide channel being longitudinally positioned on the lingual side of the elongated body;
the guide channel being operatively aligned to the guide sleeve; and
an imaging device port being positioned within the concavity.

2. The neonatal laryngeal mask airway of claim 1, wherein the at least one ventilation lumen and the at least one ventilation port comprise a pair of ventilation lumens and a pair of ventilation ports, respectively.

3. The neonatal laryngeal mask airway of claim 2, further comprising:
the imaging device port being positioned between the pair of ventilation ports; and
the imaging device port being proximally positioned to the guide sleeve.

4. The neonatal laryngeal mask airway of claim 1, wherein the imaging device port being operatively coupled to an imaging cable traversing the elongated body through an imaging cable lumen.

5. The neonatal laryngeal mask airway of claim 1, further comprising:
raised lateral ridges longitudinally traverse the lingual side of the elongated body forming the guide channel.

6. The neonatal laryngeal mask airway of claim 1, further comprising:
relative stiffness of the neonatal laryngeal mask airway being greater than relative stiffness of an intubation tube.

7. The neonatal laryngeal mask airway of claim 1, wherein the at least one ventilation lumen provides a flow of approximately 1.5 liters/min at a pressure not exceeding 20 mm of water.

8. A method of intubating a patient with a neonatal laryngeal mask air way, the method comprising:
inserting a neonatal laryngeal mask airway into the patient's pharynx, with a lingual side of the neonatal laryngeal mask airway positioned towards the patient's lingual surface;
introducing a cuff of the neonatal laryngeal mask airway into the patient's larynx and forming a seal;
aligning at least one ventilation port on the cuff with the patient's trachea;
supplying oxygenated air to the patient through the at least one ventilation port through a ventilation device by way of at least one ventilation lumen;
guiding an endotracheal tube along a guide channel longitudinally formed on the lingual side towards a guide sleeve of the cuff;
traversing the endotracheal tube through the guide sleeve and into the trachea;
switching delivery of the oxygenated air from the at least one ventilation port to the endotracheal tube; and
removing the neonatal laryngeal mask airway from the patient's mouth.

9. The method of claim 8, further comprises:
utilizing an imaging device positioned within an imaging device port in the cuff to align the endotracheal tube with the patient's trachea.
